(19) United States
(12) Patent Application Publication
(10) Pub. No.: US 2025/0032821 A1
(43) Pub. Date: Jan. 30, 2025

(54) APPARATUS COMPATIBLE WITH PHYSIOLOGICAL MEASUREMENT SYSTEMS AND ULTRASOUND BEAM GUIDANCE CONFIGURED WITH ULTRASOUND AND EEG POSTS

(71) Applicant: Sanmai Technologies, PBC, Sunnyvale, CA (US)

(72) Inventors: Tomokazu SATO, Minneapolis, MN (US); Christopher DAFT, Tucson, AZ (US); Timothy MULLEN, Hidden Meadows, CA (US)

(21) Appl. No.: 18/782,472

(22) Filed: Jul. 24, 2024

Related U.S. Application Data
(60) Provisional application No. 63/516,463, filed on Jul. 28, 2023.
(60) Provisional application No. 63/516,465, filed on Jul. 28, 2023.
(60) Provisional application No. 63/516,469, filed on Jul. 28, 2023.
(60) Provisional application No. 63/607,032, filed on Dec. 6, 2023.

(51) Int. Cl. A61N 7/00

(52) U.S. Cl. CPC A61N 7/00; A61N 2007/0026

(57) ABSTRACT

A transcranial focused ultrasound (tFUS) system integrates ultrasound and electroencephalogram (EEG) systems by providing ultrasound posts and EEG posts. A holder unit holds one or more arrays of posts that enable vertical movement and prevent lateral movement. The one or more arrays of posts include: one or more ultrasound transmitting posts, and one or more electroencephalogram (EEG) posts.

### CROSS-REFERENCE TO RELATED APPLICATIONS

This application claims priority to and the benefit of U.S. Provisional patent application titled, “APPARATUS COMPATIBLE WITH PHYSIOLOGICAL MEASUREMENT SYSTEMS AND ULTRASOUND BEAM GUIDANCE FOR NEURO NAVIGATION,” filed on Jul. 28, 2023, and having Ser. No. 63/516,463, U.S. Provisional patent application titled, “SHIELDING TECHNIQUES FOR APPARATUS COMPATIBLE WITH PHYSIOLOGICAL MEASUREMENT SYSTEMS AND ULTRASOUND BEAM GUIDANCE,” filed on Jul. 28, 2023, and having Ser. No. 63/516,465, U.S. Provisional patent application titled, “APPARATUS COMPATIBLE WITH PHYSIOLOGICAL MEASUREMENT SYSTEMS AND ULTRASOUND BEAM GUIDANCE CONFIGURED WITH ULTRASOUND AND EEG POSTS,” filed on Jul. 28, 2023, and having Ser. No. 63/516,469, and U.S. Provisional patent application titled, “CRANIAL ACOUSTIC COUPLING APPARATUS AND METHODS,” filed on Dec. 6, 2023, and having Ser. No. 63/607,032. The subject matter of these related application are hereby incorporated herein by reference.

### BACKGROUND

### Field of the Various Embodiments

This application relates to ultrasound beam guidance and deals with ultrasound beam guidance for transcranial focused ultrasound (tFUS) using electroencephalogram (EEG) measurements, more specifically an apparatus with physiological measurement systems and ultrasound beam guidance configured with ultrasound and EEG posts.

### Description of the Related Art

Transcranial focused ultrasound (tFUS) is a non-invasive neurostimulation technology utilizing low-intensity ultrasound. Compared to other techniques like magnetic or electric non-invasive brain stimulation, tFUS has better spatial resolution and safety and can reach deeper areas of the brain. FIG. 1 is a prior-art example of a tFUS system 100. Referring to FIG. 1, system 100 consists of a human skull 110 in contact with an ultrasound (US) transducer assembly 120. Transducer 120 is connected to receiver/transmitter (RX/TX) module 130. RX/TX module 130 includes various functions such as analog front end, waveform generators, amplifiers, etc. Control 140 controls system 100 and is connected to RX/TX module via connection 135. Using various stimulation parameters, tFUS can suppress or facilitate neural activity or perform tissue ablation. A drawback of system 100 is that the system does not provide feedback on the effectiveness of the stimulation.

FIG. 2 is a prior-art example of an electroencephalogram (EEG) system 200. Referring to FIG. 2, system 200 consists of one or more EEG electrodes 210 in contact with a human head or skull 110. Voltage fluctuations measured by EEG electrodes 210 are transmitted to controller 220 via wires 215. Controller 220 displays waveforms 230 measured by electrodes 210 after amplification. System 200 can be used for measuring electrical activity in response to stimulation. One drawback is that the EEG system lacks ultrasound transducers. However, ultrasound transducers can adversely affect EEG operation. Another drawback faced by systems of both FIGS. 1 and 2 is a lack of effective instruction for how to place the ultrasound and EEG components, which can lead to guesswork and ineffective operation. A further drawback is the difficulty of maintaining effective contact with a human head using traditional electrodes. As the foregoing illustrates, what is needed in the art is a way to integrate tFUS and EEG in a way that can provide effective placement and contact.

### SUMMARY

One embodiment of the present disclosure sets forth a system that includes a holder unit; and one or more arrays of posts that enable vertical movement and prevent lateral movement, the one or more arrays including one or more ultrasound transmitting posts and one or more electroencephalogram (EEG) posts.

At least one technical advantage of the disclosed techniques relative to the prior art is that, with the disclosed techniques, a tFUS and EEG system can include an individual holder unit that includes ultrasound and EEG operation concurrently. With the disclosed techniques, ultrasound transmitting posts can provide tFUS functionality while EEG posts can provide EEG electrode functionality. The posts can be thin enough to extend through hair, and can further enable vertical movement, thereby enabling superior coupling relative to existing technologies. These technical advantages provide one or more technological advancements over prior art approaches.

One embodiment of the present disclosure sets forth a method that includes: providing a holder unit; and holding, using the holding unit, one or more arrays of posts that enable vertical movement and prevent lateral movement, the one or more arrays including: one or more ultrasound transmitting posts, and one or more electroencephalogram (EEG) posts.

At least one technical advantage of the disclosed techniques relative to the prior art is that, with the disclosed techniques, a tFUS and EEG system can include an individual holder unit that includes ultrasound and EEG operation concurrently. With the disclosed techniques, ultrasound transmitting posts can provide tFUS functionality while EEG posts can provide EEG electrode functionality. The posts can be thin enough to extend through hair, and can further enable vertical movement, thereby enabling superior coupling relative to existing technologies. These technical advantages provide one or more technological advancements over prior art approaches.

One embodiment of the present disclosure sets forth an apparatus that includes: a holder unit; and one or more arrays of posts that enable vertical movement and prevent lateral movement, the one or more arrays including: one or more ultrasound transmitting posts, and one or more electroencephalogram (EEG) posts.

At least one technical advantage of the disclosed techniques relative to the prior art is that, with the disclosed techniques, a tFUS and EEG system can include an individual holder unit that includes ultrasound and EEG operation concurrently. With the disclosed techniques, ultrasound transmitting posts can provide tFUS functionality while EEG posts can provide EEG electrode functionality. The posts can be thin enough to extend through hair, and can further enable vertical movement, thereby enabling superior coupling relative to existing technologies. These technical advantages provide one or more technological advancements over prior art approaches.

### BRIEF DESCRIPTION OF THE DRAWINGS

So that the manner in which the above recited features of the various embodiments can be understood in detail, a more particular description of the inventive concepts, briefly summarized above, can be had by reference to various embodiments, some of which are illustrated in the appended drawings. It is to be noted, however, that the appended drawings illustrate only typical embodiments of the inventive concepts and are therefore not to be considered limiting of scope in any way, and that there are other equally effective embodiments.

FIG. 1 is a prior-art example of a Transcranial focused ultrasound (tFUS) system.

FIG. 2 is a prior-art example of an electroencephalogram (EEG) system.

FIG. 3 is an example tFUS and EEG system using neuro-navigation markers, according to various embodiments.

FIG. 4 shows an example tFUS and EEG system using a screen and buttons to aid neuro-navigation, according to various embodiments.

FIG. 5 shows an example tFUS and EEG system using LEDs to aid neuro-navigation, according to various embodiments.

FIGS. 6A and 6B show example placements of EEG electrodes with respect to the ultrasound field for a tFUS and EEG system, according to various embodiments.

FIG. 7 shows an example tFUS and EEG system that combines ultrasound and EEG systems, according to various embodiments.

FIG. 8 is an example of a replaceable patch unit that can be used in a tFUS and EEG system, according to various embodiments.

FIG. 9 shows an example of an ultrasound assembly for a tFUS and EEG system, according to various embodiments.

FIGS. 10A-10C show examples of a tFUS and EEG system where electrodes operate within an ultrasound pressure field, according to various embodiments.

FIGS. 11A and 11B show embodiments of ultrasound dampening in a tFUS and EEG system, according to various embodiments.

FIG. 12 illustrates an example of the electrical conductivity and insulation scheme used in the tFUS and EEG system of FIG. 7, allowing ultrasound waves to be transmitted to the head with minimal aberration, according to various embodiments.

FIGS. 13A and 13B are examples of ultrasound and EEG posts used in a post-based tFUS and EEG system, according to various embodiments.

FIG. 14A-14E show various shields that protect EEG operation in a post-based tFUS and EEG system, according to various embodiments.

FIG. 15 shows an example implementation of a tFUS and EEG system that includes an array of ultrasound posts and EEG posts shown in FIG. 13A and FIG. 13B respectively, according to various embodiments.

FIG. 16 shows a post-based tFUS and EEG system with islands of ultrasound post arrays and EEG posts, according to various embodiments.

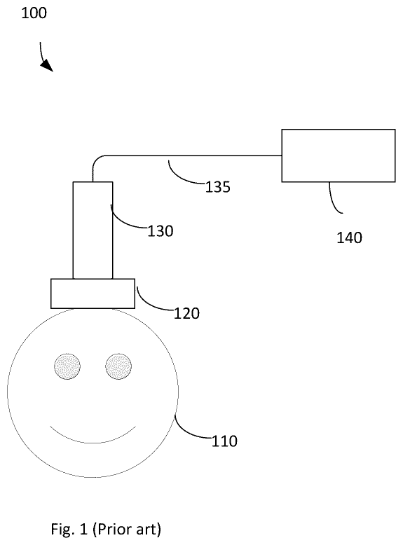

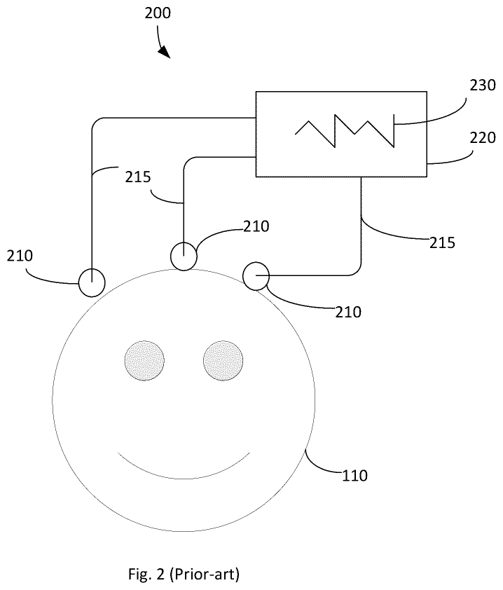

### DETAILED DESCRIPTION

In the following description, numerous specific details are set forth to provide a more thorough understanding of the various embodiments. However, it will be apparent to one of skilled in the art that the inventive concepts can be practiced without one or more of these specific details.

A transcranial focused ultrasound (tFUS) system and EEG (Electroencephalogram) is capable of integrating ultrasound (US) and EEG systems along with neuro-navigational aids to improve ultrasound beam guidance. Construction techniques with mechanical and electrical shielding materials can minimize mutual interference between ultrasound and EEG operation. To improve coupling, mess-free and user-friendly head interfacing patches, gels, and solutions are used. The head can refer to any surface of a head including without limitation the forehead, the scalp, the temples, and so on. The surface of the head or “head” can include or exclude hair, oils, and other items. A post-based tFUS system can be constructed from an array of one or more ultrasound and EEG posts to improve contact with the head. It is noted that when an ultrasound transducer is referred to, it can also be understood to be a transducer array.

### Neuro-Navigation

The tFUS and EEG system can make solid contact with the head for both US and EEG. In addition, to stimulate specific targeted brain regions, the relative location, and orientation of the tFUS system to these brain regions can be known. For this, a holder that includes tFUS and EEG components can be placed correctly on the head. To acquire high-quality EEG data, the EEG electrodes can similarly be placed in the correct locations and achieve good coupling. The complete system can include multiple independently adjustable tFUS and/or EEG units. To aid with neuro-navigation (correct placement), the tFUS and EEG systems described here use visual, audio, and haptic aids, as well as systems that work in concert to orient and register the tFUS and EEG system with the subject's head. To this end, two parts of the tFUS and EEG system include 1) navigational aids that help register the location of the tFUS and EEG systems relative to the subject's brain and 2) visual aids that help indicate to the subject or another person how to adjust the location of the systems according to the navigational info.

Neuro-navigational aids can include markers and indicators of various types. For example, neuro-navigational aids can include reflective beads or strips, as well as Light Emitting Diodes (LEDs) and other special markers that the overall system can detect using Light Detection and Ranging (LIDAR) and cameras. The neuro-navigational aids can be used with cameras or similar imaging systems to register the spatial location and orientation of the tFUS and EEG system on the subject's head. The special location and orientation can be associated with coupling of ultrasound and/or EEG. As a result, the imaging systems can be used to guide placement of the holder unit to increase ultrasound coupling and EEG coupling. A client device (such as a mobile device or a computer device), or the holder device can receive neuro-navigational detection data from a LIDAR device or a camera device. The neuro-navigational detection data can indicate a detection of the neuro-navigational aids relative to a head of a subject. The device can include an application that identifies a spatial location and orientation of the tFUS and EEG system relative to the head of the subject based on the neuro-navigational detection data. The device can also use the neuro-navigational detection data to identify characteristics of the head of the subject such as size and shape of the head. This head shape can include one or more three dimensional shapes corresponding to the subject's overall head shape. In one implementation, a smartphone-based app with an embedded camera is used to guide the placement of the tFUS and EEG system. As a visual aid, the tFUS and EEG system can activate multi-colored LEDs as feedback to indicate the correct and incorrect placement of both ultrasound and EEG components. A display device (e.g., a screen on the holder device or a client device) and/or LEDs on the holder device or another device can provide visual neuro-navigational indicators that indicate holder positioning information including which direction and orientation to move the tFUS and EEG holder. LEDs can be shaped like arrows and show how to move the system(s). In one implementation, two different sets of LEDs (spatially separated) are used to indicate the correct placement of the ultrasound and EEG, such as a left-right or up-down pair. A different set of LEDs (or other visual indicators such as user interface elements on a display device) can also indicate the coupling quality of the ultrasound and EEG. Similarly, LEDs can indicate the system's electrical or ultrasonic recording noise levels. A LIDAR system (can be internal or external to the tFUS and EEG systems) can be used to guide the placement. An internal LIDAR system with an external mirror, a camera, or a smartphone can guide the subject to place the tFUS and EEG systems. In another implementation, the tFUS and EEG system includes magnetic sensors or magnets that can be turned on and off to help alignment or spatial-registration.

Audio clues or cues can also be used as audio-based neuro-navigational indicators and aids to guide the placement. The tFUS and EEG system can include multiple speakers, which can be used for guidance. For instance, to move the tFUS and EEG system to the right, a tone can be sounded on the right speaker (with the other speakers turned off). The pitch, duration, and type of the tone can be encoded to guide correct placement or to indicate incorrect operation. Similarly, vibrations can be used for guidance. Special care is needed to ensure that neuro-navigation aids don't interfere with the system's operation.

The previous sections describe ways to register the tFUS and EEG system relative to the head and to guide the user to adjust targeting. However, there are the additional considerations of “What is the shape of the head?”, “Where in the head are the targeted brain regions?” and “How can we refine the aiming of the tFUS to the targeted brain region, especially using simultaneous EEG?” Head and brain region information can be obtained from CT (Computed tomography), fMRI (functional magnetic resonance imaging) studies, individualized MRI (magnetic resonance imaging) & fMRI subject data, individualized head-shape measurements (using calipers or similar mechanical stereotactic equipment), and individualized measurements using optical technologies such as LIDAR.

Once the tFUS and EEG system is placed on the head, it can be further adjusted for targeting using one or both ultrasound imaging and biomarkers in the EEG. In an embodiment, individualized US-based imaging from the transducers on the head includes echo time, echo strength (as well as scattering and absorption loss estimates), blood flow speed and volume measurement, tissue strain, and pulsatility, and these data and images can be used. Ultrasound-based imaging has the added advantage that head-aberration correction leads to cleaner or clearer data and images. This aberration correction can involve adjusting the timing corrections of elements of a transducer array (phase correction) and it can be done iteratively or by feeding raw data through a neural net or similar algorithm. This same head-aberration correction information can then be applied to tFUS when targeting specific brain areas. In another embodiment, one can detect a biomarker signal in the EEG from an area of interest, a nearby area, or a known area used for triangulation. The tFUS portion of the device can then be used and parameters for aiming (phasing with arrays or slight mechanical adjustments with standard transducers) fine-tuned until there is expected modulation of said biomarker. Alternatively, a tFUS waveform and target known to elicit specific EEG biomarkers, such as an evoked potential can be used to fine-tune aiming. Finally, elicited percepts, conscious or subconscious, can also be used similarly to fine-tune aiming.

The operation of the tFUS and EEG system can be described using the primary visual cortex (V1) as an example and assuming that the system has been placed in the approximately correct location using neuro-navigation and head shape information. When using ultrasound imaging of blood flow, pulsatility, and similar markers of brain activity, the system can present repeated visual stimuli at known times and in known locations of a subject's visual field and hence expect brain activity changes in specific subregions of V1 at specific times. The tFUS and EEG system can use an approximate phase correction with the transducer array to image the subregion of V1 that is expected to be activated. The tFUS and EEG system can detect a measurable, but still suboptimal, signal in response to these visual stimuli. This process can be used to generate neuro navigation indications to improve ultrasound efficacy. For example, the process can involve applying a predetermined ultrasound signal to a predetermined area, using the EEG to detect EEG biomarkers in association with a predetermined stimulus, performing a comparison of the detected EEG biomarkers to predetermined expected EEG biomarkers for the predetermined stimulus, and activating neuro-navigational indicators to indicate a direction to move the system to improve efficacy.

The tFUS and EEG system can adjust the raw data in each channel of the array such that the signals seen give a clearer image. Such algorithms for image enhancement can be simple, similar to auto-focusing by maximizing local contrast gradients in motorized lens and camera systems, or more complex, similar to having a micro-lens-fitted sensor correcting for aberrations in images using a neural net. Alternatively, or in addition, the tFUS and EEG system can use EEG biomarkers to optimize targeting feedback provided using LEDs. As before, the tFUS and EEG system can present the subject with known visual stimuli but record evoked potentials, in this case, visual evoked potentials (VEP), via EEG. Again, the tFUS and EEG system can start with an approximate starting phase correction to target this part of V1.

The tFUS and EEG system can detect a slight enhancement or suppression of the VEP depending on the tFUS targeting, waveform, and intensity. The tFUS and EEG system can also refine targeting by adjusting the ultrasound phasing parameters to see modulatory effects increase or decrease in strength, thereby determining whether phase correction is improving or worsening as it is adjusted. Alternatively, the tFUS and EEG system can omit a visual stimulus for the subject. The tFUS and EEG system can instead apply a tFUS waveform that is known to elicit a VEP reliably but is close to the threshold for intensity, focal size, or pressure gradient to do so. With the starting phase correction, the tFUS and EEG system can decline to elicit VEPs or do so only occasionally. The tFUS and EEG system can adjust the phase correction based on the improvement or worsening of the VEP reliability. Finally, beyond using an EEG biomarker like VEP, the tFUS and EEG system can use a sensory percept to adjust targeting and aiming of tFUS, in this case, a perception of a visual stimulus when none was given. With sufficiently good phase correction and aiming with tFUS, the subject can report a visual percept. However, said percept need not be consciously reportable. One can use sensitive psychophysical tasks such as asking a subject to guess in which of two time periods or locations a visual stimulus was in. Such two-alternative forced choice tasks can reliably detect biases even without a conscious percept. Improved reliability of these percepts can be utilized to adjust phase correction and targeting with tFUS.

The tFUS and EEG systems described here include holders that includes a housing or frame structure that holds ultrasound transducers and EEG electrodes in a particular arrangement. The holders can include mechanical structures and necessary circuitry that allows mounting to headbands, headsets, stereotactic arms, or other similar devices. Holders include neuro-navigation components, including time-synched or strobing LEDs, LIDAR contrast-enhancing markers, uniquely colored or patterned markers, and beads reflective of (including but not limited to) infrared light. Such markers need not be on the top of the holder, and it can be beneficial to have them on the sides of the holder.

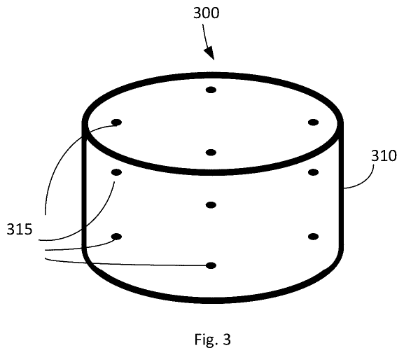

FIG. 3 shows an example tFUS and EEG system 300 using neuro-navigation markers. Referring to the figure, holder unit 310 includes neuro-navigation neuro-navigational markers 315. Neuro-navigational markers 315 are placed on the top and the side of the holder. Neuro-navigational markers 315 can be used with camera-based components to guide the placement. The camera-based components of the system can include an app executing on a smartphone, smartwatch, tablet, laptop, etc. Neuro-navigational markers 315 can include, without limitation, reflective beads, strips, LEDs, printed indicators, objects, features, and so on. The various neuro-navigational markers 315 can be visually unique and distinguishable from one another, for example, by varying size, color, shape, and so on. LED-based neuro-navigational markers 315 can include varying color, amplitude, and timing control to identify individual LEDs. Neuro-navigational markers 315 can be used with external apps or neuro-navigation systems to measure head geometry/shape and aid external neuro-navigation systems. Systems can include accelerometers, gyroscopes, etc.; neuro-navigational markers 315 and the neuro-navigation system can guide the placement.

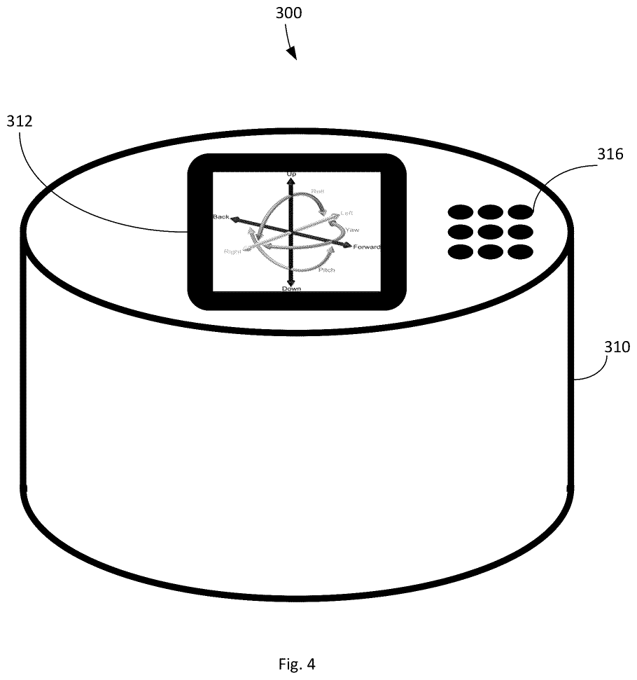

FIG. 4 shows an example tFUS and EEG system 300 using a screen and buttons to aid neuro-navigation. In FIG. 4, holder unit 310 includes a screen or display device 312 showing neuro-navigational indicators as a user interface that provides holder positioning information that indicates, without limitation, whether to move the holder unit 310 up/down, left/right, away/towards, in a roll direction, pitch direction, and/or yaw direction, along with an amplitude of adjustment expressed in color or intensity of light. In addition, an indication of US and EEG coupling, such as a bar chart, a pie chart, a percentage, a number, or another coupling indication can also shown be on display device 312. In one implementation, display device 312 is a touch screen. In another implementation, display device 312 can include buttons (e.g., push buttons. Not shown in the figures). In another implementation, buttons 316 are integrated into holder unit 310. Commands using buttons or screen allow commands to be sent to the system. Commands can be used to turn on or turn off EEG impedance measurements (which can interfere with EEG signal acquisition). Commands can be used to turn on or off ultrasound transmission of pulse waveforms. Pulse waveforms are used for alignment and differ from waveforms used for tFUS. For example, a pulse can refer to a diagnostic signal emitted by the tFUS and EEG system 300, having a relatively short duration relative to tFUS waveforms. The tFUS and EEG system 300 can capture a reflection of the pulse to perform diagnostics. The tFUS waveforms can differ from the pulse, for example, by having a longer duration, different frequency, different repeat periods, different (e.g., greater) intensity, and so on.

FIG. 5 shows an example tFUS and EEG system 300 using LEDs 318 as instructional neuro-navigational indicators to aid neuro-navigation. Paired LEDs 318 (not all LEDs are marked in the figure) and other neuro-navigational indicators can provide a user interface that shows holder positioning information. Holder positioning information can indicate, without limitation, whether to move the holder unit 310 up/down, left/right, away/towards, in a roll direction, pitch direction, and/or yaw direction, along with an amplitude of adjustment expressed in color or intensity of light. The paired LEDs 318 can be considered instructional LEDs that provide instructions for a user of the device. The system can use the neuro-navigational markers 315, to detect the systems' orientation and placement and the neuro-navigational indicators such as the paired LEDs 318 to provide holder positioning information. In an implementation, neuro-navigational markers 315, LEDs 318, etc., aid the adjustment and improve tFUS and/or EEG targeting and coupling quality. In some examples, one or more of the LEDs 318 can be used as neuro-navigational markers 315. In one implementation, colored or strobed LEDs are used and can be detected by a camera using triangulation.

### Considerations to Eliminate Aberration of US Fields and Artifacts in EEG

The key to obtaining spatial information from EEG is to have many locations of electrical recording from the head across the head. Each EEG electrode can record from a somewhat spatially-limited region of the head. An EEG electrode with low electrical-resistance contact to a large swath of the head can record a spatially-blurred and averaged signal and thus not contain much spatial information. A traditional EEG electrode is often made from materials such as silver chloride that have a poor acoustic match with soft tissue and gels. They are also often 3 mm or greater in one or more dimensions. This combination of traits makes them such that if they are in an ultrasound field, they can absorb and reflect some of that mechanical energy. The result is that the EEG electrode can “rattle” and mechanical artifact signals can appear in the EEG data. In addition, EEG electrodes can also aberrate the ultrasound field and distort the focal point or make the focal point unknown, thus leading to the modulation of unintended brain regions while missing the intended target. Due to the size of some components and the anatomy of the human brain and head, it will also not always be possible to place an EEG electrode outside the ultrasound field.

Thus, one aspect of the device is to provide new implementations of electrodes and wires relative to their traditional form. This can solve the problem of bringing multiple single (single as in independent electrically) channels of spatially-limited, local recordings of the head electrical potential signals away from the ultrasound field without affecting the ultrasound field or being affected by the ultrasound field, to the electronics used for EEG signal acquisition such as amplifiers and analog-to-digital converters. More specifically, “electrodes” can be thought of as a cohesive ensemble or assembly of components such that 1) they are in the proximity of the head area that they are recording from, 2) this electrical coupling can be aided by additional conductive gels or patches that contact the head well 3) this area of good electrical contact is spatially-limited and locations known, 4) and this single area can be seen as a single channel of signal which is then conducted by a “wire” to appropriate electronics. Said “wire” can also include several components as needed, and 1) carry the single channel of EEG information from the “electrode” to distal locations via electrical continuity, 2) insulated from surrounding areas such that other electrical signals do not leak in, and 3) bring the signals to standard wires, amplifiers, analog-to-digital converters, or other such components. As described below, these “electrodes” and “wires” can include multiple layers, sections, or materials to achieve the desired properties.

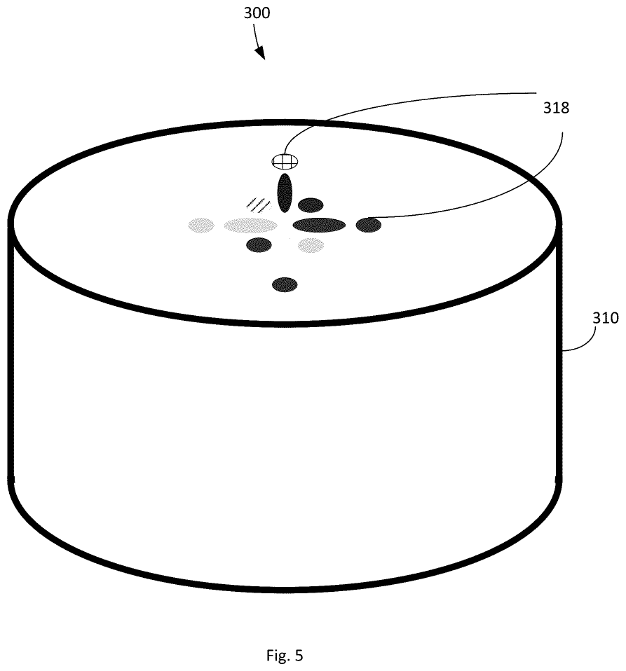

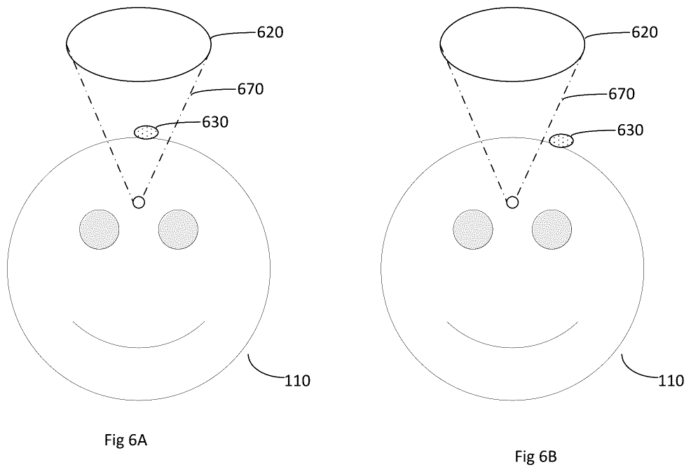

FIGS. 6A and 6B show example placements of EEG electrodes with respect to the ultrasound pressure field. In FIG. 6A, EEG electrode 630 on head 110 is placed within the ultrasound pressure field 670 generated by the ultrasound transducer or transducer array 620. In FIG. 6B, EEG electrode 630 on head 110 is placed outside the ultrasound pressure field 670 generated by the ultrasound transducer or transducer array 620. Similarly, various EEG system components (electrodes, wires, metal strands, etc.) can be placed within or outside the ultrasound pressure field. If an EEG electrode (and other EEG components) is in the path of an ultrasound wave, it can vibrate and pick up mechanical noise that is seen in the data. Even if the EEG electrode is outside the ultrasound pressure field, it can still be subject to secondary vibrations coming through a holding apparatus or from the brain (say, ultrasound coming from the other side of the head, or a reflected wave coming back out of the head). Using materials with similar acoustic properties can help reduce the vibration. Additional techniques can be employed to minimize vibration and resonance. For example, when metal strands are used, the diameter (@) of metal strands is less than the wavelength (A) of the waves used for ultrasound. In addition, the distance (separation) between metal strands is greater than the ultrasound wavelength (A). Embedding such strands in solid plastics such as LDPE can further reduce their vibrations and hence mechanical noise in the EEG data. Similar physical parameters (separation, diameter, area, thickness, acoustic impedance, speed of sound, density, etc.) can be considered when other methods (conductive gel patches, doping, metal coating, conducting film patches) are used.

EEG voltages can be of a very small magnitude (e.g., 10 to 100 μV) and are very susceptible to other forms of noise. Besides the interaction with mechanical forces leading to artifacts and noise in the EEG, electrical noise can arise from EMI or the ultrasound transducer's operation. Electrical shielding requirements for artifact-free EEG are often more stringent than those for safety. EEG operation can be insulated from the ultrasound transducer array; the insulation can often protect from both EMI (air-transmitted, without any direct conductive pathway) and direct conductive connection. Each EEG electrode operates independently, in one example embodiment, to capture the EEG signals, so the operation of each EEG electrode is insulated from each other. An EEG control system (not shown in the figures) combines EEG signals captured by individual EEG electrodes. EMI and capacitive shielding can be used to protect EEG operation. Wire mesh cages or similar conductive structures shield EEG channels, pathways, and patches to act as Faraday cages or shields. A Faraday cage's conducting material cancels any external electrical field's effect on EEG channels, paths, etc. These cages can be electrically isolated from the EEG electrode. In an embodiment, a reference EEG patch generates a reference differential signal. The reference differential signal can be used as a ground reference for the rest of the cages. An appropriate ground is used in a different embodiment, such as a battery or a capacitor. Care is taken to ensure that this grounding layer does not contact the head. In an alternate embodiment, proper shielding and isolation can be implemented on the ultrasound transducer or transducer array. Such an approach can be more efficient if multiple EEG electrodes are near a single transducer or transducer array. Particular care can be taken so that these shielding and isolation methods do not interfere with the ultrasound field or the EEG's operation.

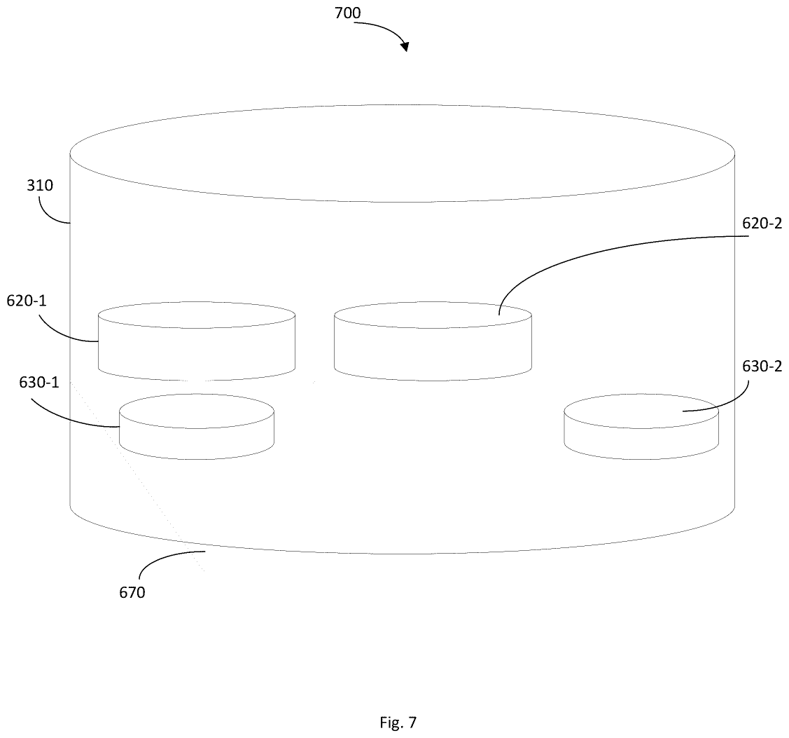

FIG. 7 shows an example of a tFUS and EEG system 700 that combines ultrasound and EEG systems. System 700 contains multiple ultrasound transducers or ultrasound transducer arrays 620 and EEG electrodes 630 in a holder unit 310. EEG electrodes 630 placed in the path of the ultrasound pressure field vibrate and pick up mechanical noise that appears in the EEG recording. These unwanted mechanical artifacts can also aberrate the ultrasound field or even block the ultrasound waves. Electrode 630-1 is placed within the pressure field 670 of transducer 620-1, while electrode 630-2 is not placed in the path of an ultrasound transducer 620. Isolating the operation of EEG electrode 630 from pressure field 670 of ultrasound transducer or transducer array 620 reduces interference. In this example, two ultrasound arrays and two electrodes are shown. System 700 can contain any number of EEG electrodes or ultrasound-emitting areas with potential spatial overlap.

The size and shape of ultrasound transducers or transducer arrays 620 and EEG electrodes 630 can be different. In one embodiment, the location, size, shape, and number of ultrasound transducer or transducer arrays 620 and EEG electrodes 630 depend on the local geometry of the head. Head geometry is obtained from existing CT and MRI studies. In an embodiment, individualized MRI data is obtained from the subject. Individualized head-shape measurements are obtained in a different embodiment using calipers or similar mechanically-based stereotactic equipment. Head-shape measurements are obtained using LIDAR and related optical technologies in another embodiment. In an embodiment, holder unit 310 includes an outer shell that can be mounted to headbands, headsets, stereotactic arms, etc. Holder unit 310 includes aids for neuro-navigation. In one implementation, multiple units within a system or multiple holders or systems can use each other (via strain gauges between units; electrical or US transmission between pairs) for additional orientation information. For example, a US transducer can be able to image EEG electrodes on the opposite side of the head via echo characteristics, mainly if an EEG electrode is surrounded by acoustically transparent, absorbing, or reflecting materials significantly different in acoustic properties. (Say the center area for the EEG electrode is acoustically matched to soft tissue but surrounded by a ring of highly acoustically-reflecting material). In an implementation, system 700 can include multiple holder units 310, with each holder unit 310 having one or more ultrasound transducers or transducer arrays 620 and electrodes 630. For example, referring to the figure, transducer or transducer array 620-2 can be in a different holder, electrode 630-2 in a second one, and transducer 620-1 & electrode 630-1 in a third holder. Holder unit 310 or holder units 310 include mounting mechanisms.

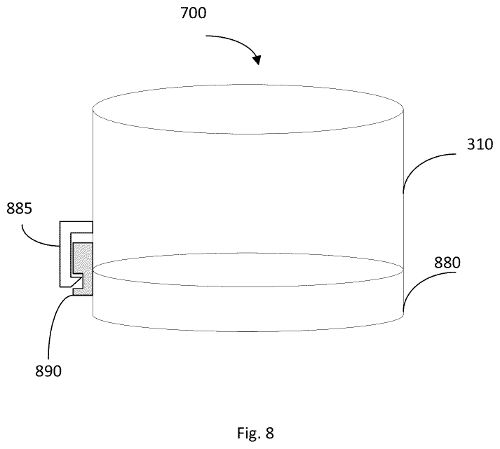

FIG. 8 is an example of a replaceable patch unit that can be used in System 700. As illustrated in FIG. 8, holder unit 310 is detachably attached to an attachment puck or replaceable patch unit 880. The replaceable patch unit 880 makes contact with the head. A latching mechanism (labeled 885 and 890 in the figure) secures the replaceable patch 880 to holder unit 310. The replaceable patch unit 880 can be loaded with patches, gels, saline solutions, etc., to improve the EEG and ultrasound interface by confirming better to the head and hair. The latching mechanism 885 and 890 makes it easy to replace patches, gels, saline solutions, etc., periodically or after every use. In an implementation, replaceable patch 880 is attached to holder unit 310 using a snap-fit button. In this example, the replaceable patch unit is shown as a single homogeneous unit, but these patches can contain islands of electrically conducting and non-conducting areas. EEG needs a spatial separation between electrodes 630 (to prevent all electrodes from detecting the same signal). In an embodiment, holder unit 310 can also contain simple heating or cooling mechanisms (e.g., resistive heating or Peltier cooling) to activate (softer or wetter to make it better conform to hair and the head and create a better EEG or ultrasound interface) or deactivate (solidification or drier/evaporation) the patch. In another embodiment, LEDs are used to activate and deactivate.

In one implementation, the patches (e.g., made of cold gum, hair pomade, or Play-Doh-like pliable substances that can be reshaped without crumbling) can have a gel with a transition temperature close to the body temperature. At room temperature, the gel can remain solid and not drip during storage and handling for application. On the head, gentle heating (from body temperature or external heating-hair dryer or built-in heating mechanisms in the holder unit 310) can liquefy the gel and make good contact with the head. In one implementation, the patches are kept at a lower temperature (e.g., in a refrigerator) until ready to use. In an implementation, holder unit 310 holds small reservoirs of gels, saline solution, etc. A small hole or perforation allows the gel/saline solution to drip into the patch unit and spread onto the head.

In one implementation, holder unit 310 includes a puncturing method to create the small hole or perforations to allow the gel/saline to drip to the head. The flow from the reservoir can be activated by gravity, mechanical, or pressure. The gel can be composed of easier-to-clean chemicals such as alcohol that evaporates like Purell® or can blend in like hair products and skin lotions after the EEG or tFUS session. In one implementation, the patch is water activated. A user dips the patch in water and applies it to the head. In one implementation, holder unit 310 can increase or reduce the pressure on the patches to improve contact with the head using turnscrews, etc. In an embodiment, patches are made of polyacrylate gel or similar super-absorbent gels that can be pressed to release some liquid for better coupling to the head or head. They can absorb much of the liquid upon release of pressure. Said replaceable patches can be used one-time or multi-times and can be obtained via one-time purchasing or subscription. An app that controls the tFUS and EEG system can also be set up to place an order after a set number of uses.

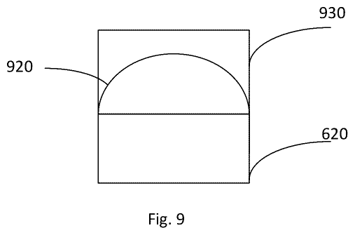

FIG. 9 shows an example of an ultrasound assembly unit per an embodiment of the invention. The ultrasound assembly unit shown in the figure can be used in System 700. As seen in FIG. 9, the coupling interface 920 and 930 can include one or more curved surfaces and interfaces for a transducer, a transducer array, a section of a transducer, a section of an array, or an individual transducer element 620. With only interface 920, these curves (either concave or convex) can help in achieving stable acoustic contact with parts of the head. When made of acoustic-impedance matched but different speed-of-sound materials, these materials can act as lensing surfaces. For example, an optional acoustic lens 920+930 changes the focus of ultrasound waves produced by transducer 620. Transducer 620 and lens 920+930 are housed within assembly unit 310, which is not shown in this figure. Lens 920+930 can be used to adjust the ultrasound beam focus and for impedance matching, timing delays, etc. In addition, while the figure shows a simple single coupling patch configuration, one can use an array of such coupling patches, akin to optical microlens arrays. Such coupling patches can not all have identical properties such as length, speed of sound, or lensing properties. Such an array of patches with optional lensing can also be used on a large transducer to adjust its focusing or on individual ultrasound array elements. In addition, instead of a single circular or rectangular grid of lenses, the coupling patch can have annular sections of different acoustic properties or one that continuously changes properties along the radius, akin to a gradient-index lens in optics.

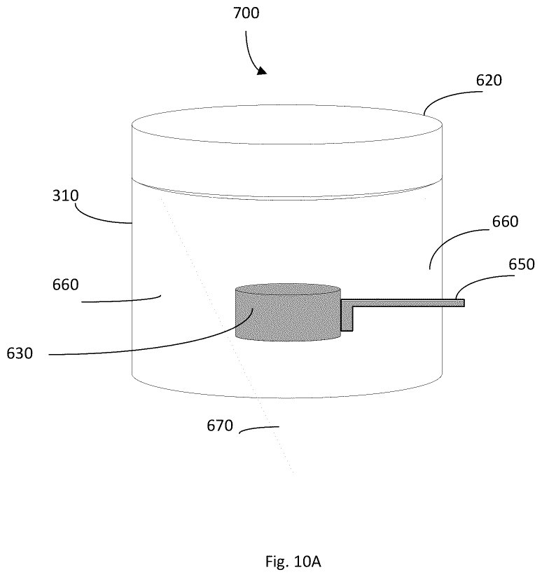

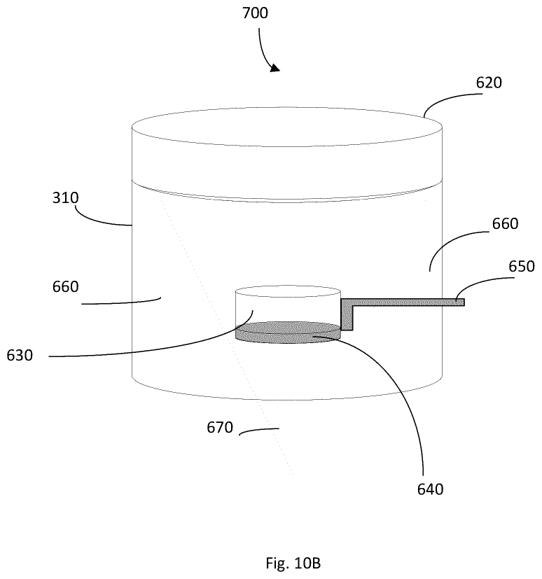

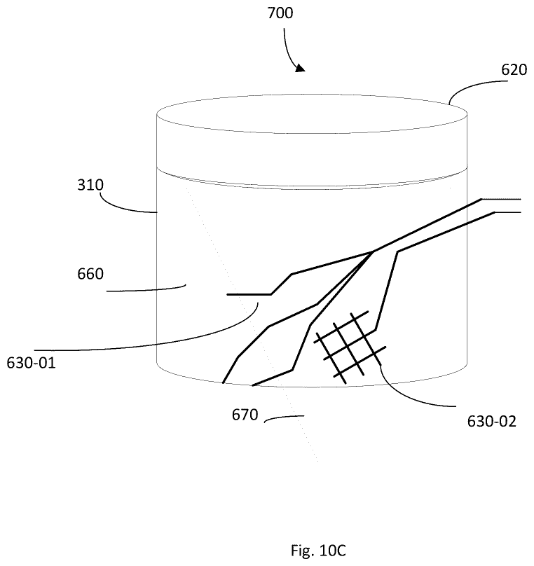

FIGS. 10A, 10B, and 10C show examples of system 700, where electrodes operate within an ultrasound pressure field. Electrode 630 is constructed so there is no ultrasound impedance mismatch with the head. This helps reduce vibrations picked up due to operating within an ultrasound pressure field and also ensures that electrode 630 does not block or aberrate the ultrasound waves. The figures show that electrode 630 operates within the ultrasound pressure field 670 of the ultrasound transducer or transducer array 620. Electrode 630 is mounted in an electrically non-conductive embedding material 660 of the holder unit 310. Embedding material 660 is electrically non-conductive and ultrasound transmissive, i.e., its acoustic properties also match that of soft tissue. Embedding material is made of materials such as silicone, plastic, etc. In FIG. 10A, electrode 630 contacts the head at the bottom in a spatially-limited area. A wire channel 650 that does not touch the head but is in electrical contact with electrode 630 brings the EEG signal through the embedding material 660 outside the ultrasound pressure field 670.

Once outside the pressure field 670, traditional electrical wires can be used. The wire channel 650, like the electrode 630, can be constructed to minimize vibrations and can be composed of multiple materials or sections. A simple example is that the electrode and wire channel can be made of a channel of saline or electrically-conductive gel for the portion in the ultrasound path. In another implementation, the electrode and wire channel are constructed of conductive material such as doped plastic or silicone. Such doped materials can have acoustic properties well-matched to tissue. In a simple example, if doping is known to cause an increase in density and speed of sound of the mixture, a base plastic or silicone with slightly less density and speed of sound than soft tissue can be used such that the resulting doped material can have similar density and speed of sound to soft tissue. In one implementation, electrode 630 is made by embedding one or more conductive channels in a non-conductive base such as plastic or silicone.

The conductive channels contain a saline solution or other conductive gels, etc. In FIG. 10B, electrode 630 is made conductive by coating it with electrically conductive thin layers or narrow channels 640 that do not aberrate the ultrasound pressure field 670. Such materials include silver-doped silicone and ITO-film-coated (Indium tin oxide) plastic. The layers or channels 640 need not be uniform as long as there is sufficient continuity to the wire channel 650. Layers or channels 640 can have gaps in a random, pseudorandom, or ordered (rows, mesh, matrix, etc.) pattern. A spatially limited bottom portion (bottom, as per the figure) of electrode 630 contacts the head. In FIG. 10C, thin-diameter fingers (630-01) or meshes of wires (630-02) in the embedding material 660 are used as the electrode 630. Diameters can be less than the wavelength of sound in the surrounding embedding material, corresponding to the wavelength of the tFUS waveform. Wires or mesh have sufficient separation and are sparse enough to have low enough effective changes in density so that they can be used without aberrating the ultrasound pressure field 670. The distance between the strands can be greater than the ultrasound wavelength. A spatially limited area that is electrically conducting makes contact with the head.

FIG. 11A and FIG. 11B show embodiments of ultrasound dampening in System 700. Ultrasound reflecting back from the head into the holder unit 310 or in various electrode, areas can cause potential mechanical rattling (especially at PRF (pulse repetition frequency)) if more traditional EEG electrodes are used, even outside the direct ultrasound pressure field. To reduce or eliminate mechanical rattling via the reflected ultrasound, the use of reflectors (air gaps or metal, for example, have high acoustic impedance mismatch with soft tissues and thus reflect the ultrasound waves, as well as convert some of the energy into radiated sound energy at the PRF) and absorbers (certain specialty rubbers and foams, for example, can absorb and dissipate the US energy as heat) are indicated. These materials can be placed behind an electrode (between the bulk of the coupling housing and the electrode, on the other side from the head) to reduce rattle from the housing; or in front of the EEG electrode as long as they do not entirely impede electrical conductivity to the head; around the EEG electrode; or in various locations of the holder or apparatus to dissipate or redirect ultrasound energy.

In FIG. 11A, electrode 630 contacts an electrically conductive but an ultrasound absorbing couplant 632 (e.g., saline-soaked rubber foam). Ultrasound absorbing couplant 632 makes contact with the head, and the incoming (from the head) ultrasound power drops rapidly as it traverses the couplant 632, and there is no electrical signal loss. In FIG. 11B, multiple layers are used. Electrode 630 is in contact with an electrically conductive gel layer 632-1, Gel layer 632-1 is in contact with a metal layer 634, metal layer 634 is in contact with gel layer 632-2, which is in contact with the head. There is no loss in electrical conductivity, but the metal layer 634 reflects back ultrasound coming from the head, reducing mechanical forces reaching electrode 630. While some layers are referred to as ‘gel layers’ other ultrasound absorbent electrically conductive materials can be used such as ESD plastics. While some layers can be referred to as metal layers, other ultrasound reflective (and/or electrically conductive) materials can be used.

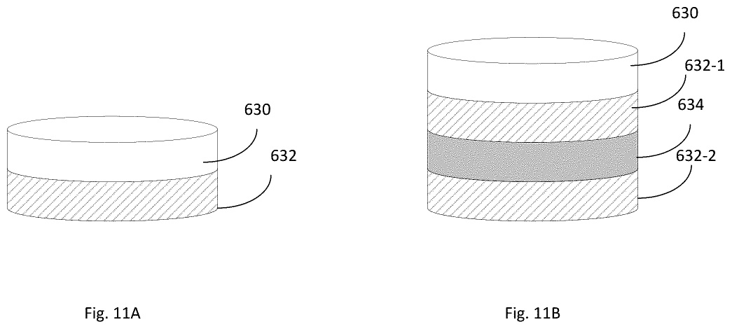

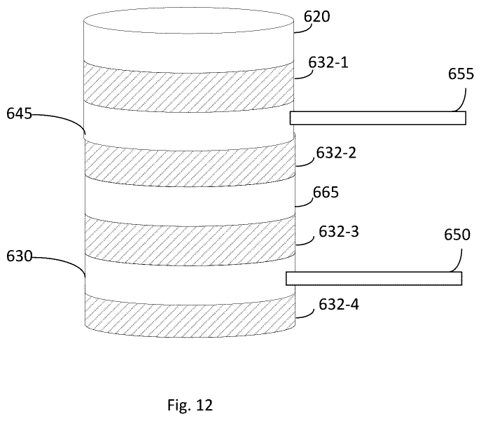

EEG voltages are of a very small magnitude (10 to 100 μV) and are very susceptible to noise. Electrical noise can arise from EMI or the ultrasound transducer's operation (for example, via capacitive coupling or direct current leakage). EEG operation can be insulated from the ultrasound transducer array; the insulation can protect from both EMI (air-transmitted, without any direct conductive pathway) and direct conductive connection. FIG. 12 illustrates an example of the electrical conductivity and insulation scheme used in System 700, allowing ultrasound waves to be transmitted to the head with minimal aberration. All layers between the transducer or transducer array 620 and the head (not shown, understood to be below layer 632-4) are ultrasound transmitting. Electrically-conducting gel layer 632-4 contacts the head and is in electrical contact with electrode 630. EEG signals are read from the head and transmitted by wire channel 650.

Gel layer 632-3 separates electrode 630 and an electrically insulating layer 665. Insulating layer 665 provides electrical isolation between EEG electrical system (electrode 630, wire channel 650, etc.) and the ultrasound transducer or array 620. Gel layer 632-2 separates insulating layer 665 and an electrically conductive shielding layer 645. Shielding layer 645 is connected to a grounding wire 655. Shielding layer 645 can act as Faraday cage or shield, for example, using a wire mesh cage or similar conductive structure to provide electrical isolation from electrical noise caused by ultrasound vibrations incident and/or reflected. Grounding wire 655 is electrically isolated from the head and EEG electrical system and is connected to an appropriate electrical ground. Shielding layer 645 shunts EMI and currents from transducer 620. The combination of shielding layer 645 and insulating layer 665 provides the best reduction in electrical artifacts for both EMI and direct conductive connection. However, in some implementations, using only one shielding layer 645 or insulating layer 665 can suffice. Ultrasound array 620 is separated from the shielding layer 645 by a gel layer 632-1. Other arrangements using conductive shielding 645 and insulating layer 665 are possible. The ultrasound-transmitting, electrically-conducting layers (630, 645) can be implemented using techniques described in FIGS. 10A, B, and C. Gel layers 632-1˜4 are ultrasound transmitting. If used, gel layer 632-4 can be electrically conducted so EEG signals reach electrode 630.

### Post-Based Implementation to Transmit Ultrasound and EEG

The head introduces a number of complexities to both transcranial ultrasound methods (both for imaging and modulation of brain activity) and for EEG recordings. The head is highly aberrating to ultrasound fields and can be corrected for. The head and head also smear EEG signals, making them significantly lower in signal-to-noise ratio, temporal resolution, and spatial resolution compared to ECoG recordings from on top or just below the dura mater. These difficulties, unfortunately, can be handled using better hardware (ultrasound arrays and electrode arrays) and algorithms (to correct for the head and head). However, in addition to those issues, the rigidity and various local curvatures of the head, combined with the relative thinness of the head, makes solid coupling of systems to the head difficult. Small dimples and protrusions can be particularly problematic. Furthermore, the presence of hair can be a significant barrier to good ultrasound and electrical coupling. To better address these issues for both ultrasound and EEG, we describe a system using posts, in post holders, to transmit ultrasound and electrical signals for use in combined tFUS and EEG.

The posts are designed to be capable of getting between hair or individually conform to and contact better the skin and any small dimples or roughness on the skin. In one example the posts can include a semi-stiff brush (a 2D array of bristles/posts) where the individual bristles are utilized for good coupling acoustically and electrically. This approach is compatible with various types of transducer and transducer array geometries. While each post can have some give and adjustability in the direction orthogonal to the head (up and down), for the ultrasound-transmitting posts, their lateral location contacting the head can remain rigid, constant and known. This is because changing their location along the head can change where they effectively are transmitting ultrasound to and recording ultrasound from. This information can be utilized for accurate head aberration correction algorithms. While modern technology can detect such deflections in the posts, the cost and complexity of doing so in the tight confines of the system can be difficult. However, it is acceptable for the holders of these posts to be mounted on a flexible backing. This is because if the holders and posts are sufficiently rigid, the backing's curvature can be measured in any number of methods including via a network of built-in strain gauges or optical methods, and hence the post tip locations are known. The 2D curvature of the backing is relatively easy to measure. This is simpler than attempting to determine the precise 3D location of the tips of each and every post, especially in the hair, if the posts or holders had slack or were pliable. EEG posts and ultrasound transmitting posts can be electrically conductive based on at least one of: patches of conductive gel, patches of conductive film, doping, metal coating, or metal strands embedded in the one or more EEG posts. EEG posts and ultrasound transmitting posts can be electrically conductive based on metal strands that have a diameter less than the ultrasound wavelength, and a distance between the strands that is greater than the ultrasound wavelength. EEG posts and ultrasound transmitting posts can have conductive gel coatings bonded to the one or more EEG posts. In some examples, EEG posts and ultrasound transmitting posts can be constructed to be acoustically similar so that the same post or same post construction can be used for EEG and/or ultrasound operation.

FIG. 13A and FIG. 13B are examples of ultrasound and EEG posts used in a post-based tFUS and EEG system. FIG. 13A shows an example of an ultrasound post-assembly. FIG. 13B is similar to FIG. 13A but has an electrically conductive assembly supporting EEG functionality. FIG. 13A shows a sectional view of the post-based system 1300 with a holder unit 1305. A single post 1340 in a single holder 1305 is displayed in this example. Holder unit 1305 provides mechanical and structural support for the post. Holder unit 1305 provides electrical insulation and ultrasound dampening to keep the operation between various posts interference-free. In one embodiment, an array of holder units 1305 (along with the corresponding ultrasound transducer array elements and EEG electronics) is mounted on a flexible backing and allows System 1300 to conform better to the curvature of the human head. The material used for Holder unit 1305 or between holder units is selected to dampen vibrations to reduce cross-talk between ultrasound channels.

Holder unit 1305 can be made of one or more materials or layers to provide the necessary properties. For example, holder 1305 can have an outer silicone layer to provide electrical and ultrasound isolation and an inner layer made of metal or ceramic to provide rigid mechanical support to the post 1340. Post 1340 can be of single construction or contain internal channels or structures such as 1350. Spring layer 1330 allows for a degree of movement of the post along its length, orthogonal to the head, allowing for better contact even with local dimples or bumps on the head. The structure of holder 1305 prevents the lateral displacement of the post 1340. Spring layer 1330 can be compliant and have acoustic properties matched to the post and soft tissue. Additional details regarding spring layer 1330 are discussed below. An additional optional assembly unit 1310, described later, can be placed below post 1340. Note that spring layer 1330 can also extend in between post 1340 and assembly unit 1310 or below assembly unit 1310. Alternatively, assembly unit 1310 can simply be a continuation of the spring layer 1330. The bottom of holder 1305 can be enclosed or opened such that the bottom surface of spring layer 1330 or assembly unit 1310 can be exposed instead. The figure depicts a single specific configuration for illustrative purposes only and is not meant to be a limitation of the aforementioned minor configuration details and differences.

Additional considerations for EEG-supporting post components 1345/1355 make it electrically conductive. Strands of metal (or metal wires) fully embedded within post 1345 are unlikely to vibrate and pick up mechanical vibration artifacts. Films and gel coatings are unlikely to pick up mechanical vibrations when strongly bonded to the bulk of post 1345. Similarly, saline and ionic gels in channel area 1355 are unlikely to pick up mechanical vibrations as their acoustic properties are similar to Post 1345. Similarly, EEG operation is shielded from EMI (electromagnetic interference) and other electrical interference. Shielding techniques employed will be described in more detail. For EEG-compatible post 1345, spring layer 1330 can also be conductive electrically, i.e., doped silicone that is conductive electrically. In one example embodiment, for a matrix array with a 1 mm pitch, post 1340/1345 outer diameter is 0.7 mm, while holder 1305 is of a square shape with later dimensions of 1.0 mm with a wall thickness of 0.15 mm at the top where the post 1340 slides through, with a slightly less wall thickness below where the spring layer 1330 is placed to allow for its expansion or compression as needed.

In one embodiment, an array of holder units 1305 are placed on a transducer or transducer array. When placed on a matrix array, aligning each holder unit 1305 and each post to a single array element is particularly suitable. In another embodiment, embedded within Holder unit 1305 is assembly unit 1310 (1315 in FIG. 3B). Assembly unit 1310 can house a transducer array element, control, analog front end, and other signal conditioning systems. Attached to the assembly unit is a spring or cushioning layer 1330. In one implementation, spring layer 1330 is constructed from silicone. In another implementation, spring layer 1330 is made of rubber. Post 1340 is embedded within spring layer 1330. Post 1340 is made of a stiff material such as plastic and prevents lateral movement (i.e., maintain X-Y location). Embedding posts 1340 within the spring layer 330 allows for a slight vertical offset (Z-offset) and solid contact with the head. The Z-offset created by spring layer 1330 can affect phasing and targeting due to the change in distance to the head. However, these changes can be compensated for by transmitting a test pulse and assessing the reflected received signals. An advantage of a post-based tFUS system is that these posts can get between hair, make better contact with the skin, and compensate for small dimples or skin roughness. In one embodiment, the pitch of the posts 1340 can match the pitch of individual ultrasound transducer elements (within assembly 1310), i.e., one post for each ultrasound transducer array element. Post 1340 can be made of solid material.

Alternatively, in one embodiment, included at the tip of each post 1340 is a couplant assembly unit 1350. Couplant assembly unit 1350 can include gel tips, rubber, or silicone and make contact with the head. Couplant assembly 1350 can be flat or convex shaped to allow for good contact with the head. Couplant assembly 1350 can contain a channel connecting to a gel or saline reservoir outside holder 1305. The diameter of Couplant assembly 1350 is the same as that of the inner diameter of Post 1340. In one example embodiment, posts are hollow and concentric with channels for liquids or gels. In a different embodiment, couplant assembly unit 1350 includes nubs (not shown explicitly) with small refillable reservoirs for liquids or gels. When post 1340 is pressed against the head (pressure or spring activated) or due to gravity, a small amount of fluid can ooze out to form a couplant on the head. This reduces the acoustic impedance mismatch caused by any air pockets between post 1340 and head 110. Materials used for post 1340, couplant assembly unit 1350, and spring layer 1330 are acoustically matched. Assembly unit 1310, spring layer 1330, post 1340, and couplant assembly unit 1350 are collectively referred to as an ultrasound post module or ultrasound post. As mentioned, multiple such modules can be used in a single tFUS and EEG system.

FIG. 13B shows electrically conductive post-assembly to support EEG functionality. It includes assembly unit 1315, which is electrically conductive or contains EEG-related electronics, an electrically conductive post 1345, and an electrically conductive couplant unit 1355. Alternatively, only assembly unit 1315 and post 1345 can be electrically conductive and transmits head EEG signals to the EEG electronics. In another embodiment, post 1345 can not be electrically conductive, but assembly unit 1316 and couplant unit 1355 remains electrically conductive and transmits the head EEG signals to unit 1315 or beyond to the rest of the EEG electronics. The functionality of the components of the post-assembly shown in FIG. 13B are similar to those in FIG. 13A. Assembly unit 1315, spring layer 1330, post 1345, and couplant assembly unit 355 are collectively referred to as the EEG post module. In one embodiment, assembly units 1310 & 1315, posts 1340 & 345, and couplant units 1350 & 1355 are made of materials with similar acoustic impedance and match the acoustic properties of the ultrasound post. They are constructed using the same materials in a different embodiment. They can be constructed using non-conducting materials, and Assembly unit 1310, post 1340, and couplant 1350 are made electrically conductive by using one of the following, without limitation: applying patches of electrically conductive gel (with salt ions, Saline, or ionic gels), by doping the material, metal coating of the surface, applying patches of conducting films; strands of metal, metal wires.

The EEG post can be made electrically conductive by either making the surface conductive or by embedding conductive channels (channels not shown in the figures) within the non-conductive material of the EEG post. EEG posts can be made conductive using a combination of the above; for instance, it can be made conductive using a combination of conductive gels or films and metal wires or strands. Special care is taken to ensure no mutual interference in the operation of the ultrasound and EEG posts. In an implementation, couplant unit 1355 comprises a conductive gel or conductive solution in electrical contact with the conductive portion of post 1345.

FIG. 13A and FIG. 13B show a cylindrical (circular or ellipse) post-based tFUS system. To make better contact with the head or to ease manufacturing, posts can be made with triangular cross-sections, rectangular cross-sections, etc. The post-based tFUS system can comprise a mixture of triangular, rectangular, and cylindrical posts in one implementation. In some implementations, depending on the ultrasound wavelength or the characteristics of the transducer or transducer array, posts can occupy more than one mm2. In some examples, the posts can occupy a size of up to nine mm2.

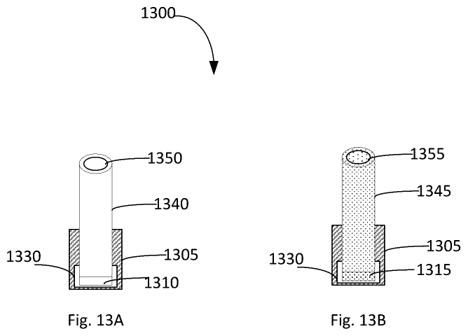

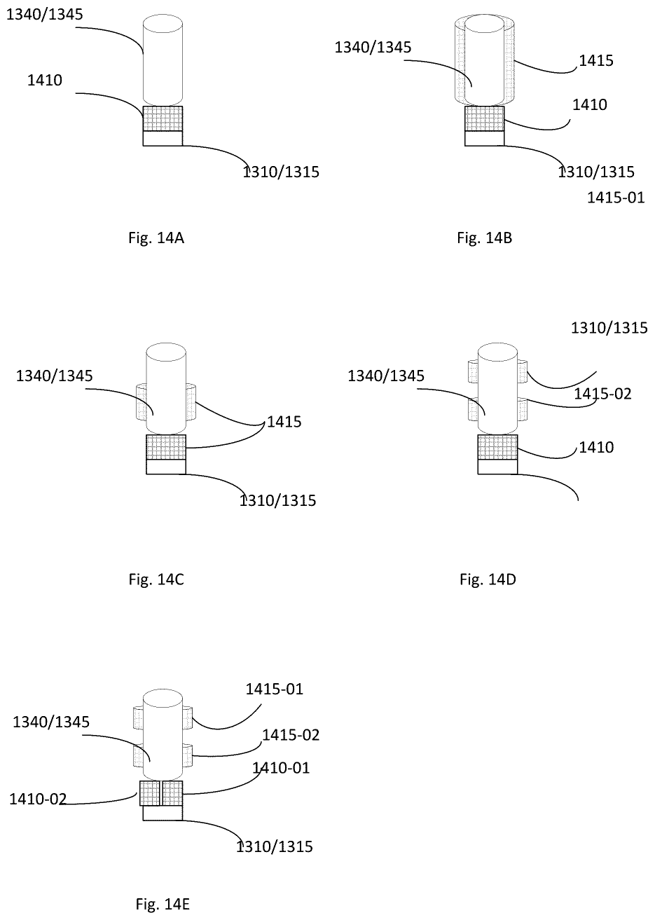

FIG. 14A-14E show various shields that protect EEG operation in a post-based tFUS system. In FIG. 14A, a shielding layer 1410 separates post 1340/1345 and assembly unit 1310/1315. Shielding layer 1410 is electrically isolated from post 1340/1345 and assembly unit 1310/1315 and acts as a Faraday cage. The ground connections are not explicitly shown in these figures. FIG. 14B shows the post 1340/1345 circumference shielded by a shielding layer 1415 in addition to shielding layer 1410 between post 1340/1345 and assembly unit 1310/1315. FIG. 14C is similar to FIG. 14B, but the circumference shielding 1415 for post 1340/1345 partially covers the length of post 1340/1345. FIG. 14D shows two circumference shielding segments, 1415-01 and 1415-02 shields post 1340/1345. More than two segments of circumference shielding 1415 can be used for shielding post 1340/1345. FIG. 14E is similar to FIG. 14D; here shielding layer between post 1340/3145 and assembly unit 1310/1315 is split into two segments, 1410-01 and 1410-02. These figures are meant to be informative of the possible geometries of shielding layers in a post-based system and are not meant to be limiting. These shielding layers can be implemented using the approaches and techniques disclosed in describing FIG. 10, where the materials' physical characteristics and implementations are chosen to prevent mutual interference between ultrasound and electrical signals and components carrying them.

The use of posts, combined with independent holders, between which further electrical shielding or ultrasound isolation (via reflection or absorption) can be done, allows for a unique method to reduce the effects of known aberrations and artifacts in 1) ultrasound transmission and imaging and 2) EEG recording, while solving the complexity of achieving 3) good acoustic and 4) low electrical impedance coupling on the 5) head and even through 6) hair.

In addition to the previous considerations of eliminating US aberrations and EEG artifacts applied to the general electrode and transducer configurations, the post-based implementation allows for further discussion of specific implementations.

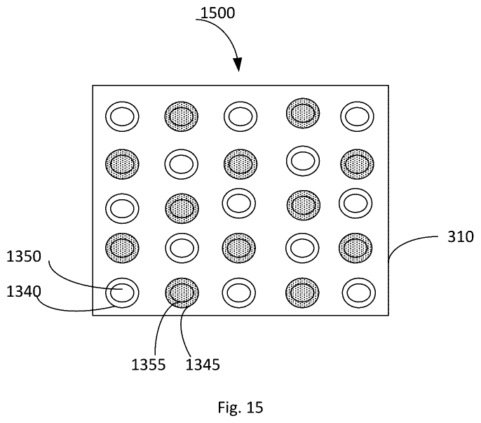

FIG. 15 shows an example implementation of a tFUS system 1500 including an array of ultrasound posts and EEG posts shown in FIG. 13A and FIG. 13B, respectively. FIG. 15 shows the top view of System 1500 and can include, for example, a 5×5 array of ultrasound posts and EEG posts, or an array of another selected predetermined size. In one embodiment, posts are arrayed into one mm2 grids (the figure is not to scale). In this example, ultrasound and EEG posts are embedded alternately into the holder unit 310. Other arrangements of ultrasound and EEG posts are possible. As seen in the figure, System 1500 looks like and functions like a stiff hairbrush, with the posts acting like the bristles of a hairbrush. As posts 1340/1345 and their holders are made of stiff material, no lateral movement is possible. Spring layer 1330 allows for vertical movement. The posts that are arrayed on one mm2 grids are small enough to make good contact between hair and account for the curvature of the head 110. Air gaps or spaces between posts help to prevent cross-talk of ultrasound signals between posts. In an embodiment, posts are separated using vibration-dampening material such as sponges or similar material.

The figure illustrates System 1500 to be flat and rectangular shaped. System 400 can be constructed to be curved and concaved (an elongated U-shaped like a headband) to make better contact with the head. In an implementation, the headband-shaped System 1500 has one or more extensions perpendicular (or angled) to the headband. In an embodiment, System 1500 is constructed like a helmet to cover most of the head and can extend over the forehead and temple regions of the head 110. System 1500 can be constructed using other structures like visors, half-helmet, half-headband, etc.

In an embodiment, ultrasound and EEG posts are constructed to support both ultrasound and EEG functionality. In one implementation, the functionality can be supported simultaneously. In another implementation, the functionality of the post can be configured at run time, for example, using an electrical switch. In one embodiment, all post 1340 are of the same length. In another embodiment, posts 1340 are different lengths and can be used as delay lines (effectively a lens or to adjust ultrasound focusing). System 1500 can include islands of EEG and ultrasound posts in one embodiment. In addition, System 1500 can be constructed on a flexible substrate. In one implementation, multiple flexible patches of System 1500 are mounted on a stiffer substrate to allow further conformity to the individual shape of the head. In these cases, the ultrasound coupling can be maintained between the ultrasound transducer or transducer array element, the ultrasound posts, and the head. With ultrasound arrays, the arrays themselves can be mounted on a flexible substrate and coupled to a System 1500 on a flexible substrate. Alternatively, each holder unit and post can be mounted individually to each array element. In another implementation, smaller patches of stiffer arrays and stiffer System 1500 are coupled to each other, with these smaller patches on a flexible backing. In another implementation, an ultrasound transducer or array with a stiff emitting surface can have a softer coupling interface (e.g., silicone or hydrogel or a conforming water bag in plastic) and allows for the interfacing with a System 1500 built on a flexible substrate.

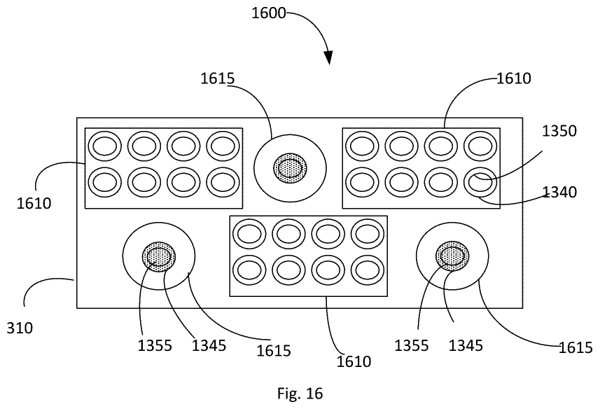

FIG. 16 shows a post-based tFUS system 1600 with islands of ultrasound post arrays and EEG posts. Referring to FIG. 16, an array of ultrasound posts are organized into an island 1610. Similarly, EEG posts operate within islands 1615. The islands can be electrically isolated from one another. This example shows three EEG islands 1615 and three ultrasound islands 1610. Other configurations are possible with a different number of ultrasound islands 1610, and EEG islands 1615. Ultrasound island 1610 can include a different number of ultrasound posts and also can be configured as an array of a different dimension (2×4 array shown in this example). Similarly, EEG island 1615 can include a different number of EEG posts (one EEG post is shown). Organizing System 1600 as islands of ultrasound and EEG reduces interference between the ultrasound arrays, between ultrasound arrays and EEG posts, and between EEG posts. Islands 1610/1615 can include shielding and insulation (not shown in the figure). EEG islands 1615 and ultrasound islands 1610 in an embodiment are based on the head's geometry. Head information can be obtained from CT, fMRI studies, individualized MRI & fMRI subject data, individualized head-shape measurements (using calipers or similar mechanical stereotactic equipment), and individualized measurements using optical technologies such as LIDAR. In an embodiment, individualized US-based imaging from the transducers on the head includes echo time, echo strength (as well as scattering and absorption loss estimates), blood flow speed and volume measurement, tissue strain, and pulsatility, and these images can be used. While FIG. 16 is shown as a post-based example with ultrasound post arrays and EEG posts, the islands can alternatively refer to electrically isolated areas of a bottom of a holder unit 310 and/or replaceable patch unit 880 with electrodes and ultrasound transmission areas as discussed with respect to other figures.

At least one technical advantage of the disclosed techniques relative to the prior art is that, with the disclosed techniques, a tFUS and EEG system can include an individual holder unit that includes ultrasound and EEG operation concurrently. With the disclosed techniques, ultrasound transmitting posts can provide tFUS functionality while EEG posts can provide EEG electrode functionality. The posts can be thin enough to extend through hair, and can further enable vertical movement, thereby enabling superior coupling relative to existing technologies. These technical advantages provide one or more technological advancements over prior art approaches.

Aspects of the subject matter described herein are set out in the following numbered clauses.

1. In some embodiments, a system comprises one or more arrays of posts comprising one or more ultrasound transmitting posts, and one or more electroencephalogram (EEG) posts, wherein a respective post of the one or more arrays of posts enables vertical movement and prevents lateral movement, and a holder unit that holds the one or more arrays of posts.

2. The system of clause 1, wherein at least one of the one or more EEG posts and the one or more ultrasound transmitting posts are electrically conductive.

3. The system of clauses 1 or 2, wherein at least one of the one or more EEG posts and the one or more ultrasound transmitting posts are electrically conductive based on at least one of patches of conductive gel, patches of conductive film, doping, metal coating, or metal strands embedded in the one or more EEG posts.

4. The system of any of clauses 1-3, wherein at least one of the one or more EEG posts and the one or more ultrasound transmitting posts are electrically conductive based on metal strands comprising a diameter less than the ultrasound wavelength, and the distance between the strands is greater than the ultrasound wavelength.

5. The system of any of clauses 1-4, wherein at least one of the one or more EEG posts and the one or more ultrasound transmitting posts comprise conductive gel coatings bonded to the at least one of the one or more EEG posts and the one or more ultrasound transmitting posts.

6. The system of any of clauses 1-5, wherein the one or more EEG posts and the one or more ultrasound transmitting posts are constructed to be acoustically similar.

7. The system of any of clauses 1-6, wherein a respective post of the one or more arrays of posts are connected to a spring layer that enables the vertical movement.

8. The system of any of clauses 1-7, wherein a respective ultrasound transmitting post of the one or more ultrasound transmitting posts is connected to a spring layer that is acoustically matched to at least one of the respective ultrasound transmitting post and soft tissue.

9. The system of any of clauses 1-8, wherein a respective EEG post of the one or more EEG posts is connected to a conductive spring layer comprising doped silicon or doped rubber.

10. The system of any of clauses 1-9, wherein the posts of the one or more arrays are separated using at least one of air gaps or vibration-damping material.

11. The system of any of clauses 1-10, wherein at least a subset of the one or more ultrasound transmitting posts are arranged in an ultrasound post array in a first area, at least a subset of the one or more EEG posts are arranged in an EEG array in a second area, and first area is electrically isolated from the second area.

12. The system of any of clauses 1-11, wherein the holder unit comprises a concave surface, and the one or more arrays of posts extend from the concave surface.

13. The system of any of clauses 1-12, wherein at least a subset of the posts comprise internal channels that deliver couplant materials.

14. The system of any of clauses 1-13, further comprising an assembly unit that houses one or more of a transducer array element, a control system, and an analog front end, wherein the assembly unit is connected to a spring layer.

15. The system of any of clauses 1-14, further comprising a couplant assembly unit that makes contact with a scalp of a subject, wherein the couplant assembly unit comprises at least one of gel tips, rubber, or silicone, and the couplant assembly unit is acoustically, electrically, or acoustically and electrically matched to at least one post of the one or more arrays of posts.

16. The system of any of clauses 1-15, wherein the couplant assembly unit is flat or convex shaped, and wherein the couplant assembly unit is connected to one or more internal channels or one or more reservoirs for delivery of couplant.

17. The system of any of clauses 1-16, further comprising one or more wire mesh cages that shield EEG pathways for the one or more EEG posts.

18. In some embodiments, a method comprises providing a holder unit, and holding, using the holding unit, one or more arrays of posts comprising one or more ultrasound transmitting posts, and one or more electroencephalogram (EEG) posts, wherein a respective post of the one or more arrays of posts enables vertical movement and prevents lateral movement.

19. The method of clause 18, wherein the one or more EEG posts are electrically conductive.

20. The method of clauses 18 or 19, wherein a respective post of the one or more arrays of posts are connected to a spring layer that enables the vertical movement.

21. The method of any of clauses 18-20, wherein a respective ultrasound transmitting post of the one or more ultrasound transmitting posts is connected to a non-conductive spring layer that is acoustically matched to at least one of the respective ultrasound transmitting post and soft tissue.

22. The method of any of clauses 18-21, wherein a respective EEG post of the one or more EEG posts is connected to a conductive spring layer comprising doped silicon or doped rubber.

23. The method of any of clauses 18-22, wherein the posts of the one or more arrays are separated using at least one of air gaps or vibration-damping material.

24. The method of any of clauses 18-23, wherein at least a subset of the one or more ultrasound transmitting posts are arranged in an ultrasound post array in a first area, at least a subset of the one or more EEG posts are arranged in an EEG array in a second area, and first area is electrically isolated from the second area.

25. The method of any of clauses 18-24, wherein the holder unit comprises a concave surface, and the one or more arrays of posts extend from the concave surface.

26. In some embodiments, an apparatus comprises one or more arrays of posts comprises one or more ultrasound transmitting posts, and one or more electroencephalogram (EEG) posts, wherein a respective post of the one or more arrays of posts enables vertical movement and prevents lateral movement, and a holder unit that holds the one or more arrays of posts.

Any and all combinations of any of the claim elements recited in any of the claims and/or any elements described in this application, in any fashion, fall within the contemplated scope of the present invention and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments can be embodied as a system, method or computer program product. Accordingly, aspects of the present disclosure can take the form of an entirely hardware embodiment, an entirely software embodiment (including firmware, resident software, micro-code, etc.) or an embodiment combining software and hardware aspects that can all generally be referred to herein as a “module,” a “system,” or a “computer.” In addition, any hardware and/or software technique, process, function, component, engine, module, or system described in the present disclosure can be implemented as a circuit or set of circuits. Furthermore, aspects of the present disclosure can take the form of a computer program product embodied in one or more computer readable medium(s) having computer readable program code embodied thereon.

Any combination of one or more computer readable medium(s) can be utilized. The computer readable medium can be a computer readable signal medium or a computer readable storage medium. A computer readable storage medium can be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium can be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions can be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors can be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams can represent a module, segment, or portion of code, which comprises one or more executable instructions for implementing the specified logical function(s). It should also be noted that, in some alternative implementations, the functions noted in the block can occur out of the order noted in the figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure can be devised without departing from the basic scope thereof, and the scope thereof is determined by the claims that follow.

What is claimed is:

1. A system comprising:
one or more arrays of posts comprising: one or more ultrasound transmitting posts, and one or more electroencephalogram (EEG) posts, wherein a respective post of the one or more arrays of posts enables vertical movement and prevents lateral movement; and
a holder unit that holds the one or more arrays of posts.

2. The system of claim 1, wherein at least one of the one or more EEG posts and the one or more ultrasound transmitting posts are electrically conductive.

3. The system of claim 2, wherein at least one of the one or more EEG posts and the one or more ultrasound transmitting posts are electrically conductive based on at least one of: patches of conductive gel, patches of conductive film, doping, metal coating, or metal strands embedded in the one or more EEG posts.

4. The system of claim 1, wherein at least one of the one or more EEG posts and the one or more ultrasound transmitting posts are electrically conductive based on metal strands comprising a diameter less than the ultrasound wavelength, and the distance between the strands is greater than the ultrasound wavelength.

5. The system of claim 1, wherein at least one of the one or more EEG posts and the one or more ultrasound transmitting posts comprise conductive gel coatings bonded to the at least one of the one or more EEG posts and the one or more ultrasound transmitting posts.

6. The system of claim 1, wherein the one or more EEG posts and the one or more ultrasound transmitting posts are constructed to be acoustically similar.

7. The system of claim 1, wherein a respective post of the one or more arrays of posts are connected to a spring layer that enables the vertical movement.

8. The system of claim 1, wherein a respective ultrasound transmitting post of the one or more ultrasound transmitting posts is connected to a spring layer that is acoustically matched to at least one of: the respective ultrasound transmitting post and soft tissue.

9. The system of claim 1, wherein a respective EEG post of the one or more EEG posts is connected to a conductive spring layer comprising doped silicon or doped rubber.

10. The system of claim 1, wherein the posts of the one or more arrays are separated using at least one of: air gaps or vibration-damping material.

11. The system of claim 1, wherein at least a subset of the one or more ultrasound transmitting posts are arranged in an ultrasound post array in a first area, at least a subset of the one or more EEG posts are arranged in an EEG array in a second area, and first area is electrically isolated from the second area.

12. The system of claim 1, wherein the holder unit comprises a concave surface, and the one or more arrays of posts extend from the concave surface.

13. The system of claim 1, wherein at least a subset of the posts comprise internal channels that deliver couplant materials.

14. The system of claim 1, further comprising an assembly unit that houses one or more of: a transducer array element, a control system, and an analog front end, wherein the assembly unit is connected to a spring layer.

15. The system of claim 1, further comprising a couplant assembly unit that makes contact with a scalp of a subject, wherein the couplant assembly unit comprises at least one of gel tips, rubber, or silicone, and the couplant assembly unit is acoustically, electrically, or acoustically and electrically matched to at least one post of the one or more arrays of posts.

16. The system of claim 15, wherein the couplant assembly unit is flat or convex shaped, and wherein the couplant assembly unit is connected to one or more internal channels or one or more reservoirs for delivery of couplant.

17. The system of claim 15, further comprising one or more wire mesh cages that shield EEG pathways for the one or more EEG posts.

18. A method comprising:
providing a holder unit; and
holding, using the holding unit, one or more arrays of posts comprising: one or more ultrasound transmitting posts, and one or more electroencephalogram (EEG) posts, wherein a respective post of the one or more arrays of posts enables vertical movement and prevents lateral movement.

19. The method of claim 18, wherein the one or more EEG posts are electrically conductive.

20. The method of claim 18, wherein a respective post of the one or more arrays of posts are connected to a spring layer that enables the vertical movement.

21. The method of claim 18, wherein a respective ultrasound transmitting post of the one or more ultrasound transmitting posts is connected to a non-conductive spring layer that is acoustically matched to at least one of: the respective ultrasound transmitting post and soft tissue.

22. The method of claim 18, wherein a respective EEG post of the one or more EEG posts is connected to a conductive spring layer comprising doped silicon or doped rubber.

23. The method of claim 18, wherein the posts of the one or more arrays are separated using at least one of: air gaps or vibration-damping material.

24. The method of claim 18, wherein at least a subset of the one or more ultrasound transmitting posts are arranged in an ultrasound post array in a first area, at least a subset of the one or more EEG posts are arranged in an EEG array in a second area, and first area is electrically isolated from the second area.

25. The method of claim 18, wherein the holder unit comprises a concave surface, and the one or more arrays of posts extend from the concave surface.

26. An apparatus comprising:
one or more arrays of posts comprising: one or more ultrasound transmitting posts, and one or more electroencephalogram (EEG) posts, wherein a respective post of the one or more arrays of posts enables vertical movement and prevents lateral movement; and
a holder unit that holds the one or more arrays of posts.
